(19) United States
(12) Patent Application Publication
(10) Pub. No.: US 2024/0243327 A1
(43) Pub. Date: Jul. 18, 2024

(54) NAPHTHALENE DIIMIDE COMPOUND AND REDOX FLOW BATTERY CONTAINING THE SAME

(71) Applicant: Korea Advanced Institute of Science and Technology, Yuseong-gu (KR)

(72) Inventors: Hye Ryung BYON, Yuseong-gu (KR); Mu-Hyun BAIK, Yuseong-gu (KR); Vikram SINGH, Yuseong-gu (KR); Seongmo AHN, Yuseong-gu (KR); Seongyeon KWON, Yuseong-gu (KR)

(21) Appl. No.: 18/224,886

(22) Filed: Jul. 21, 2023

(30) Foreign Application Priority Data
Jan. 17, 2023 (KR) 10-2023-0007418

(51) Int. Cl. H01M 8/18; C07D 471/06

(52) U.S. Cl. CPC H01M 8/188; C07D 471/06; H01M 2300/0002

(57) ABSTRACT

The present disclosure relates to a novel naphthalene diimide compound, an electrolyte solution for a redox flow battery containing the same, and a redox flow battery containing the same. The naphthalene diimide compound according to one embodiment exhibits a robust redox behavior, has high solubility in water, and has an advantage of being able to operate at neutral pH. In addition, since the naphthalene diimide compound according to one embodiment may improve the energy density by combining two electrons, it is useful as an additive for an electrolyte solution for a redox flow battery.

### CROSS-REFERENCE TO RELATED APPLICATIONS

This application claims priority to Korean Patent Application No. 10-2023-0007418, filed Jan. 17, 2023. The entire contents of the above-listed application are hereby incorporated by reference for all purposes.

### TECHNICAL FIELD

The following disclosure relates to a naphthalene diimide compound and a redox flow battery containing the same.

### BACKGROUND

Redox flow batteries (RFBs) are a promising technology for developing scalable, inexpensive, and safe solutions for grid-scale energy storage, and current state-of-the-art flow batteries using vanadate have an energy density of 25 Wh⁻¹. Meanwhile, since intrinsic chemical properties such as a redox potential and solubility cannot be changed, improvements of vanadate battery characteristics are fundamentally limited, and thus, development of charge carriers is required.

As charge carriers, organic molecules may be attractive candidates because there is an inexhaustible supply of carbon, hydrogen, nitrogen, and oxygen on the earth. In addition, properties of organic molecules may be varied over a wide range by changing a molecular structure and using a functional group.

An ideal charge carrier should exhibit a fully reversible redox reaction without degradation, be highly soluble in water, and operate at neutral pH. In order to increase stability, robust redox activity may be achieved by using an aromatic system capable of implementing delocalization of charge. However, the non-polar nature of the aromatic compound has a problem of lowering solubility in water, and a radical intermediate thereof is highly reactive, which may cause an undesirable side reaction and degradation of battery performance.

Meanwhile, the pH of the electrolyte solution during battery operation deserves special attention. Highly acidic or basic conditions are often encountered because many organic systems use proton-coupled electron transfer to achieve charge neutrality. It should be noted that an excessive amount of OH⁻ or H⁺ ions may corrode cell components or cause undesirable reactions, and ultimately may diminish a volumetric energy density and long-term cyclability.

### SUMMARY

An embodiment of the present disclosure is directed to providing an ammonium derivative compound having a naphthalene diimide scaffold as a novel compound.

Another embodiment of the present disclosure is directed to providing an electrolyte solution for a redox flow battery capable of improving performance of the redox flow battery, and a redox flow battery containing the same.

In one general aspect, there is provided a naphthalene diimide compound represented by the following Chemical Formula 1 as a novel compound:

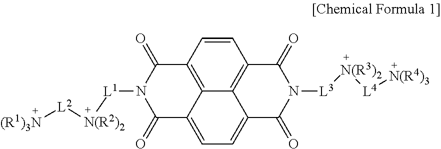

in Chemical Formula 1,
L₁, L₂, L₃, and L₄ are each independently a bond or a C₁₋₂₀ alkylene group; and
when R₁, R₂, R₃, and R₄ are each independently hydrogen, a C₁₋₂₀ alkyl group, a C₂₋₂₀ alkenyl group, a C₅₋₂₀ cycloalkyl group, or a C₆₋₂₀ aryl group, or R₁, R₂, R₃, or R₄ is an alkyl group, R₁, R₂, R³, and R₄ may be linked together with the N atoms to which they are bonded to form a heterocycloalkyl.

In one embodiment, L₁, L₂, L₃, and L₄ may be each independently a bond or a C₁₋₁₀ alkylene group; and when R₁, R₂, R³, and R₄ are each independently hydrogen, a C₁₋₁₀ alkyl group, a C₂₋₁₀ alkenyl group, a C₅₋₁₀ cycloalkyl group, or a C₆₋₁₀ aryl group, or R₁, R₂, R₃, or R₄ is an alkyl group, R₁, R₂, R₃, and R₄ may be linked together with the N atoms to which they are bonded to form a heterocycloalkyl.

In one embodiment, L₁, L₂, L₃, and L₄ may be each independently a bond or a C₁₋₅ alkylene group; and R₁, R₂, R₃, and R₄ may be each independently hydrogen, a C₁₋₅ alkyl group, or a C₂₋₅ alkenyl group.

In one embodiment, R₁, R₂, R₃, and R₄ may be each independently a C₁₋₃ alkyl group.

In one embodiment, the naphthalene diimide compound may be a compound represented by the following Chemical Formula 1-1:

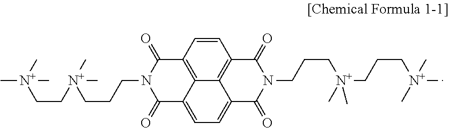

In another general aspect, an electrolyte solution for a redox flow battery contains the naphthalene diimide compound according to one embodiment.

In one embodiment, the electrolyte solution for a redox flow battery may further contain a solvent, and the solvent may be an aqueous solvent, a non-aqueous solvent, an ionic liquid, or a mixture of two or more thereof.

The aqueous solvent may be one or more selected from the group consisting of potassium chloride (KCl), sodium chloride (NaCl), potassium hydroxide (KOH), sodium hydroxide (NaOH), potassium bromide (KBr), zinc chloride (ZnCl₂), ammonium chloride (NH₄Cl), sulfuric acid (H₂SO₄), hydrochloric acid (HCl), phosphoric acid (H₃PO₄), and methanesulfonic acid (CH₃SO₄); the non-aqueous solvent may be one or more selected from the group consisting of dimethylacetamide (DMA), acetonitrile (MeCN), dimethyl carbonate, diethyl carbonate, methyl ethyl carbonate, dimethylformamide, propylene carbonate, ethylene carbonate, N-methyl-2-pyrrolidone, fluoroethylene carbonate, gamma-butyrolactone, dimethyl sulfoxide, diethylene glycol dimethyl ether, triethylene glycol dimethyl ether, tetraethylene glycol dimethyl ether, acetone, acetylacetone, 1,4-dioxane, 1,2-dimethoxyethane, dichloromethane, 1,2-dichloroethane, nitrobenzene, nitromethane, tetrahydrofuran, 2-methyltetrahydrofuran, 2,4-dimethyltetrahydrofuran, methoxybenzene, diglyme, triglyme, tetraglyme, 4-methyl-2-pentanone, propionitrile, butyronitrile, isobutyronitrile, benzonitrile, sulfolane, dimethylthioformamide, methyl acetate, ethyl acetate, ethanol, and methanol; and the ionic liquid may be an ammonium, imidazolium, morpholinium, phosphonium, piperidinium, pyridinium, pyrrolidinium, or sulfonium-based ionic liquid.

In one embodiment, the electrolyte solution for a redox flow battery may further contain a supporting electrolyte.

In one embodiment, the electrolyte solution for a redox flow battery may further contain an electrode active material, and the electrode active material may contain a 2-valent to 5-valent vanadium-based compound. In this case, the 2-valent to 5-valent vanadium-based compound may comprise one or two or more selected from the group consisting of V²⁺, V³⁺, VO²⁺, and VO₂⁺.

In still another general aspect, a redox flow battery contains the electrolyte solution for a redox flow battery according to one embodiment.

In one embodiment, the redox flow battery comprises: a positive electrode cell comprising a positive electrode and a positive electrode electrolyte solution; a negative electrode cell comprising a negative electrode and a negative electrode electrolyte solution; and a separator disposed between the positive electrode cell and the negative electrode cell, and the negative electrode electrolyte solution may be the electrolyte solution for a redox flow battery according to one embodiment.

Other features and aspects will be apparent from the following detailed description, the drawings, and the claims.

### BRIEF DESCRIPTION OF THE DRAWINGS

FIG. 1 is a diagram showing the results of analyzing solubility of compounds of Example and Comparative Examples in water and 1.5 M KCl.

FIG. 2 is a diagram showing the results of analyzing cyclic voltammetry (CV) of the compound of Example 1 in a 1 M KCl solution at a scan rate of 50 mV·s⁻¹.

FIG. 3 is a schematic diagram of a battery in which a 1.5 M KCl aqueous solution is used as a negative electrode electrolyte solution (negolyte) and a 3I⁻/I₃⁻ redox pair is used as a positive electrode electrolyte solution (posolyte) to perform a flow battery test using the compound of Example 1.

FIG. 4 shows two successive charging plateaus of galvanostatic profiles of a redox flow battery (RFB), in which c₁ and c₂ processes are the first plateau (0 to 50% stage-of-charge (SOC)) and the second plateau (50 to 100% stage-of-charge (SOC)), respectively.

FIG. 5 is a diagram showing the results of analyzing UV-Vis absorption spectra, near infrared radiation (NIR) absorption spectra, electron paramagnetic resonance (EPR) spectra, ¹H NMR, and solution pH by collecting each of negative electrode electrolyte solutions after SOC for chemical analysis of the RFB.

FIG. 6 is a diagram showing an interaction mechanism of the compound of Example 1 during a cathodic reaction.

FIG. 7 is a diagram showing CV curves of the compound of Example 1 and I⁻/I₃⁻ at a scan rate of 50 mV·s⁻¹.

FIG. 8 is a diagram showing the results of analyzing an open-circuit voltage (OCV) and an area-specific resistance (ASR) of a separator.

FIG. 9 is a diagram showing the results of evaluating coulombic efficiency (CE), voltage efficiency (VE), and energy efficiency (EE) of the RFB.

FIG. 10 is a diagram showing galvanostatic curves for the 2^{nd}, 250^{th}, and 500^{th} cycles at 25 mA·cm⁻².

FIG. 11 is a diagram showing a cycle profile for 500 cycles (45 days).

### DETAILED DESCRIPTION OF EMBODIMENTS

Embodiments disclosed in the present specification may be modified into many different forms and the technology according to one embodiment is not limited to the embodiments described below. Furthermore, in the entire specification, unless explicitly described otherwise, “comprising”, “comprising”, “containing”, or “having” any components will be understood to imply the inclusion of other components rather than the exclusion of any other components, and does not exclude elements, materials, or processes which are not further listed.

A numerical range used in the present specification comprises upper and lower limits and all values within these limits, increments logically derived from a form and span of a defined range, all double limited values, and all possible combinations of the upper and lower limits in the numerical range defined in different forms. As an example, when a content of a composition is limited to 10% to 80% or 20% to 50%, a numerical range of 10% to 50% or 50% to 80% should also be interpreted as described in the present specification. Unless otherwise specifically defined in the present specification, values out of the numerical range that may occur due to experimental errors or rounded values also fall within the defined numerical range.

Hereinafter, in the present specification, unless otherwise specifically defined, “about” may be considered a value within 30%, 25%, 20%, 15%, 10%, or 5% of a stated value.

The term “alkylene group” used in the present specification refers to a linear or branched diradical of a carbon saturated bond, and comprises an alkylene group substituted with an arbitrary substituent such as halogen.

The term “alkyl group” used in the present specification refers to a linear or branched radical of a carbon saturated bond, and may be substituted with an arbitrary substituent such as halogen. The term “alkenyl group” used in the present specification refers to a linear or branched carbon chain radical containing one or more e carbon unsaturated bonds (double bonds), and may be substituted with an arbitrary substituent such as halogen.

Ring substituents such as the terms “cycloalkyl group”, “aryl group”, and “heterocycloalkyl group” used in the present specification are not limited to a bridged ring, a spiro ring, or a fused ring.

In the present specification, the term “negative electrode electrolyte solution (negolyte, anolyte, or negative electrode electrolyte)” is an electrolyte solution in which a negative electrode active material is dissolved, and the term “positive electrode electrolyte solution (posolyte, catholyte, or positive electrode electrolyte)” is an electrolyte solution in which a positive electrode active material is dissolved. The positive electrode active material refers to a redox pair dissolved in a positive electrode electrolyte solution, and means that charging occurs when the redox pair changes to a higher one of two oxidation states, that is, when oxidation occurs. The negative electrode active material refers to a redox pair dissolved in a negative electrode electrolyte solution, and means that charging occurs when the redox pair changes to a lower one of two oxidation states, that is, when reduction occurs.

In one embodiment, a novel naphthalene diimide compound containing four ammonium ions is provided, and the naphthalene diimide compound according to one embodiment exhibits a robust redox behavior and may have improved solubility in water through derivatization. In addition, the naphthalene diimide compound is effective for application to batteries because it has an advantage of being able to operate under neutral pH conditions.

Since the naphthalene diimide compound according to one embodiment may nullify the radical character while forming a cluster of dimers or tetramers, unwanted chemical reactions and battery performance degradation may be effectively prevented by lowering a highly reactive radical character. In addition, as the naphthalene diimide compound according to one embodiment forms a cluster of dimers or tetramers so that a compound structure is three-dimensionally formed through intramolecular interactions, crossover between pores of a membrane is prevented, and at the same time, excessive stacking of the compound is not formed, such that high solubility may be maintained.

Hereinafter, a naphthalene diimide compound according to one embodiment, an electrolyte solution containing the same, and a battery containing the same will be described in detail.

A naphthalene diimide compound provided in one embodiment is represented by the following Chemical Formula 1:

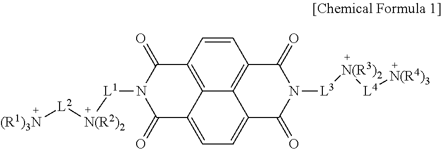

in Chemical Formula 1,
L₁, L₂, L₃, and L₄ are each independently a bond or a C₁₋₂₀ alkylene group; and
when R₁, R₂, R₃, and R₄ are each independently hydrogen, a C₁₋₂₀ alkyl group, a C₂₋₂₀ alkenyl group, a C₅₋₂₀ cycloalkyl group, or a C₆₋₂₀ aryl group, or R₁, R₂, R₃, or R₄ is an alkyl group, R₁, R₂, R³, and R₄ may be linked together with the N atoms to which they are bonded to form a heterocycloalkyl.

A plurality of R₁, R₂, R₃, and R₄ are all connected to a nitrogen atom, and the plurality of R₁, R₂, R₃, and R₄ are independent substituents, which may be different from each other or identical to each other. The plurality of R¹ linked to the nitrogen atom may be formed by being linked together with the nitrogen atom to which they are bonded to form a 3- to 20-membered or 3- to 10-membered heterocycloalkyl, and the same is applied to R₂, R₃, and R₄.

In one embodiment, L₁, L₂, L₃, and L₄ may be each independently a bond or a C₁₋₁₀ alkylene group; and when R₁, R₂, R₃, and R₄ are each independently hydrogen, a C₁₋₁₀ alkyl group, a C₂₋₁₀ alkenyl group, a C₅₋₁₀ cycloalkyl group, or a C₆₋₁₀ aryl group, or R₁, R₂, R₃, or R₄ is an alkyl group, R₁, R₂, R₃, and R₄ may be linked together with the N atoms to which they are bonded to form a heterocycloalkyl.

In one embodiment, L₁, L₂, L₃, and L₄ may be each independently a bond or a C₁₋₅ alkylene group; and R₁, R₂, R₃, and R₄ may be each independently hydrogen, a C₁₋₅ alkyl group, or a C₂₋₅ alkenyl group.

In one embodiment, L₁, L₂, L₃, and L₄ may be each independently a C₁₋₁₅ alkylene group, a C₁₋₁₀ alkylene group, a C₁₋₆ alkylene group, a C₁₋₅ alkylene group, a C₂₋₆ alkylene group, a C₂₋₅ alkylene group, a methylene group, an ethylene group, a propylene group, or a butylene group.

In one embodiment, R₁, R₂, R₃, and R₄ may be each independently a C₁₋₁₅ alkyl group, a C₁₋₁₀ alkyl group, a C₁₋₆ alkyl group, a C₁₋₅ alkyl group, a C₁₋₃ alkyl group, an ethyl group, or a methyl group, a C₂₋₁₅ alkenyl group, a C₂₋₁₀ alkenyl group, a C₂₋₆ alkenyl group, a C₂₋₅ alkenyl group, or a C₂₋₃ alkenyl group, or a C₅₋₁₀ cycloalkyl group, a C₅₋₆ cycloalkyl group, a C₆₋₁₀ aryl group, or a C₆₋₈ aryl group.

In one embodiment, specifically, the naphthalene diimide compound may be a compound represented by the following Chemical Formula 1-1:

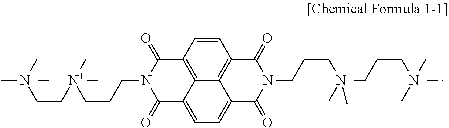

In another embodiment, there is provided an electrolyte solution for a redox flow battery containing the naphthalene diimide compound according to one embodiment.

In one embodiment, the electrolyte solution for a redox flow battery may further contain a solvent, and the solvent may be, for example, an aqueous solvent, a non-aqueous solvent, an ionic liquid, or a mixture of two or more thereof.

Any solvent may be applied without limitation as long as it is a solvent known in the art disclosed in the present specification, and non-limiting examples thereof are listed below.

The aqueous solvent may contain, for example, any one or more of Li⁺, Na⁺, K⁺, and NH₄⁺ as a cation, or may contain any one or more of Cl⁻, I⁻, Br⁻, OH⁻, and NO₃⁻ as an anion. Specifically, the aqueous solvent may be one or more selected from the group consisting of potassium chloride (KCl), sodium chloride (NaCl), potassium hydroxide (KOH), sodium hydroxide (NaOH), potassium bromide (KBr), zinc chloride (ZnCl₂), ammonium chloride (NH₄Cl), sulfuric acid (H₂SO₄), hydrochloric acid (HCl), phosphoric acid (H₃PO₄), and methanesulfonic acid (CH₃SO₄).

The non-aqueous solvent may be one or more selected from the group consisting of dimethylacetamide (DMA), acetonitrile (MeCN), dimethyl carbonate, diethyl carbonate, methyl ethyl carbonate, dimethylformamide, propylene carbonate, ethylene carbonate, N-methyl-2-pyrrolidone, fluoroethylene carbonate, gamma-butyrolactone, dimethyl sulfoxide, diethylene glycol dimethyl ether, triethylene glycol dimethyl ether, tetraethylene glycol dimethyl ether, acetone, acetylacetone, 1,4-dioxane, 1,2-dimethoxyethane, dichloromethane, 1,2-dichloroethane, nitrobenzene, nitromethane, tetrahydrofuran, 2-methyltetrahydrofuran, 2, 4-dimethyltetrahydrofuran, methoxybenzene, diglyme, triglyme, tetraglyme, 4-methyl-2-pentanone, propionitrile, butyronitrile, isobutyronitrile, benzonitrile, sulfolane, dimethylthioformamide, methyl acetate, ethyl acetate, ethanol, and methanol.

The ionic liquid may be an ammonium, imidazolium, morpholinium, phosphonium, piperidinium, pyridinium, pyrrolidinium, or sulfonium-based ionic liquid.

In one embodiment, the electrolyte solution may have a viscosity of 10 cps to 2,000 cps when measured at 25° C.

In one embodiment, the electrolyte solution for a redox flow battery may further contain a supporting electrolyte. The supporting electrolyte may contain one or more of an alkylammonium salt, a lithium salt, a sodium salt, and a potassium salt.

In one embodiment, the electrolyte solution for a redox flow battery may further contain an electrode active material, and the electrode active material is dissociated in the electrolyte solution to be oxidized and reduced to cause a reaction, through which the redox flow battery is charged and discharged. The electrode active material is not particularly limited, and an active material commonly used in the technical field disclosed in the present specification may be used. For example, the electrode active material may be V, Fe, Cr, Cu, Ti, Sn, Zn, Br, and the like. As a specific example, the electrode active material may be a 2-valent to 5-valent vanadium-based compound, and the 2-valent to 5-valent vanadium-based compound may comprise one or two or more selected from the group consisting of V²⁺, V³⁺, VO²⁺, and VO₂⁺.

A concentration of the naphthalene diimide compound contained in the electrolyte solution for a redox flow battery according to one embodiment is not particularly limited, and may be, for example, 0.01 M to 5.0 M, 0.1 M to 4.0 M, 0.1 M to 3.0 M, 0.1 M to 2.0 M, 0.5 M to 3.0 M, 0.5 M to 4.0 M, or 0.5 M to 2.0 M.

In still another embodiment, there is provided a redox flow battery comprising the electrolyte solution for a redox flow battery according to one embodiment.

The redox flow battery may comprise: a positive electrode cell comprising a positive electrode and a positive electrode electrolyte solution; a negative electrode cell comprising a negative electrode and a negative electrode electrolyte solution; and a separator disposed between the positive electrode cell and the negative electrode cell, and the negative electrode electrolyte solution may be the electrolyte solution for a redox flow battery.

As the separator that may be comprised in the redox flow battery according to one embodiment, any separator may be adopted without limitation as long as it is a material capable of transferring ions, and may be selected from materials commonly used in the technical field disclosed in the present specification. For example, the separator may contain a polymer having cationic conductivity, or may be a separator in which a polymer having cationic conductivity is provided in pores of a porous body. As a specific example, the polymer having cationic conductivity may be a hydrocarbon-based polymer, a partial fluorine-based polymer, or a fluorine-based polymer.

As an example, a polymer having cationic conductivity may comprise at least one of Nafion, sulfonated polyetheretherketone (SPEEK), sulfonated (polyetherketone) (SPEK), poly (vinylidene fluoride)-graft-poly (styrene sulfonic acid) (PVDF-g-PSSA), and sulfonated poly (fluorenyl ether ketone).

The porous body may comprise at least one of polyimide (PI), nylon, polyethyleneterephtalate (PET), polytetrafluoro ethylene (PTFE), polyethylene (PE), polypropylene (PP), poly(arylene ether sulfone) (PAES), and polyetheretherketone (PEEK).

In one embodiment, a thickness of the separator may be, for example, 10 μm to 200 μm or 20 μm to 100 μm.

The positive electrode electrolyte solution may comprise one or more selected from 1, 1′-dimethylferrocene, a salt of (ferrocenylmethyl) trimethylammonium, a salt of bis((3-trimethylammonio) propyl) ferrocene, 5,10-dimethyl-5,10-dihydrophenazine, 5,10-bis(2-methoxyethyl)-5,10-dihydrophenazine, (2,2,6,6-tetramethylpiperidin-1-yl)oxyl (TEMPO), 4-hydroxy-(2,2,6,6-Tetramethylpiperidin-1-yl)oxyl (4-hydroxy-TEMPO), 4-amino-(2,2,6,6-Tetramethylpiperidin-1-yl)oxyl (4-amino-TEMPO), a salt of N,N,N-2,2,6,6-heptamethylpiperidinyloxy-4-ammonium (TMA-TEMPO), a salt of N1,N1,N1,N3,N3,2,2,6,6-nonamethyl-N3-(piperidinyloxy)propane-1,3-bis(ammonium), a salt of 4-[3-(trimethylammonio)propoxy]-2,2,6,6-tetramethylpiperidine-1-oxyl (TMAP-TEMPO), N-ethylphenothiazine, N-ethyl-3,7-dimethylphenothiazine, N-ethyl-3,7-dimethoxyphenothiazine, N-(2-(2-methoxyethoxy) ethyl)phenothiazine, 3,7-dimethoxy-N-(2-(2-methoxyethoxy) ethyl)phenothiazine, 3,7-bis(2-(2-methoxyethoxy) ethoxy)-N-(2-(2-methoxyethoxy)ethyl)phenothiazine, 2,3-dimethyl-1,4-dimethoxybenzene, 2,5-dimethyl-1,4-dimethoxybenzene, 1,4-di-tert-butyl-2-methoxy-5-(2-methoxyethoxy)benzene, 1,4-di-tert-butyl-2-methoxy-5-(2-(2-methoxyethoxy)ethoxy)benzene, 1,4-di-tert-butyl-2,5-bis(2-methoxyethoxy)benzene, 1,4-di-tert-butyl-2,5-bis (2-(2-methoxyethoxy)ethoxy) benzene, a salt of N-(2,3-bis(diisopropylamino)cycloprop-2-en-1-ylidene)-N-isopropylpropan-2-aminium, and a salt of 2,3-bis(diisopropylamino)-1-(methylthio)cycloprop-2-en-1-ylium.

By adopting a negative electrode electrolyte solution containing the naphthalene diimide compound having high redox stability, a low reduction potential, and excellent solubility as an organic active material for a negative electrode electrolyte solution, the redox flow battery may have a high potential difference and a high energy density. Accordingly, the redox flow battery may be suitable for applications requiring high capacity and high output, such as an electric vehicle, in addition to existing applications such as a mobile phone and a portable computer, and may be used in a hybrid vehicle or the like in combination with an existing internal combustion engine, fuel cell, or supercapacitor. In addition, the redox flow battery may be used for all other applications requiring high output and high voltage.

In addition, the redox flow battery according to one embodiment may be widely used for stationary energy storage capable of storing renewable energy such as solar light in electrical energy storage (EES) and smart cities.

Hereinafter, Examples and Experimental Examples will be described in detail below. However, Examples and Experimental Examples to be described below are only to illustrate some embodiments, and the technology described in the present specification is not construed as being limited thereto.

### EXAMPLE 1

### Preparation of 4A⁴⁺-NDI

Step 1: Preparation of 2,7-bis (3-(dimethylamino)propyl)benzo[lmn] [3,8]phenanthroline-1,3,6,8(2H,7H)-tetraone (NMez-Pr-NDI)

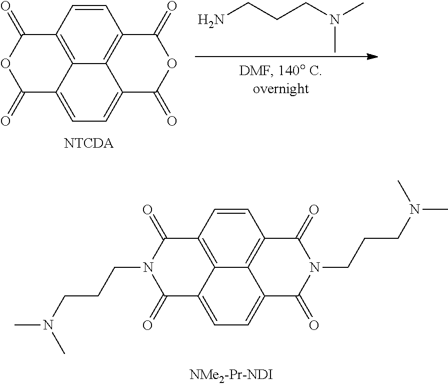

3.0 g of 1,4,5,8-naphthalenetetracarboxylic dianhydride (NTCDA) was added to 20 mL of anhydrous DMF with stirring in a 100 mL round bottom flask in an argon atmosphere. 5 equivalents of 3-(dimethylamino)-1-propylamine was added thereto with stirring. The resulting mixture was refluxed at 140° C. overnight. The mixture was cooled to room temperature, and then green needle-shaped crystals of NMe₂-Pr-NDI were collected by filtration. The crystals were washed with 20 mL of DMF, 50 mL of acetone, and 20 mL of diethyl ether, and the washed crystals were dried at 100° C. under vacuum, thereby obtaining 4.0 g of a target compound (yield: 92%).

¹H NMR of NMe₂-Pr-NDI: (CDCl₃, 300 MHZ): δ 8.75 (s, 4H, Ar—H), δ 4.26 (t, 4H, —N_{imide}—CH₂—), δ 2.43 (t, 4H, —N_{imide}—CH₂—), δ2.23 (s, 12H, —N(CH₃)₂), δ1.92 (quint, 4H, —CH₂—CH₂—CH₂)

Step 2: N¹, N¹′-((1,3,6,8-tetraoxo-1,3,6,8-tetrahydrobenzo[lmn] [3,8]phenanthroline-2,7-diyl)bis(propane-3,1-diyl))bis(N¹, N¹,N³,N³,N³-pentamethylpropane-1,3-diaminium (4A⁴⁺-NDI)

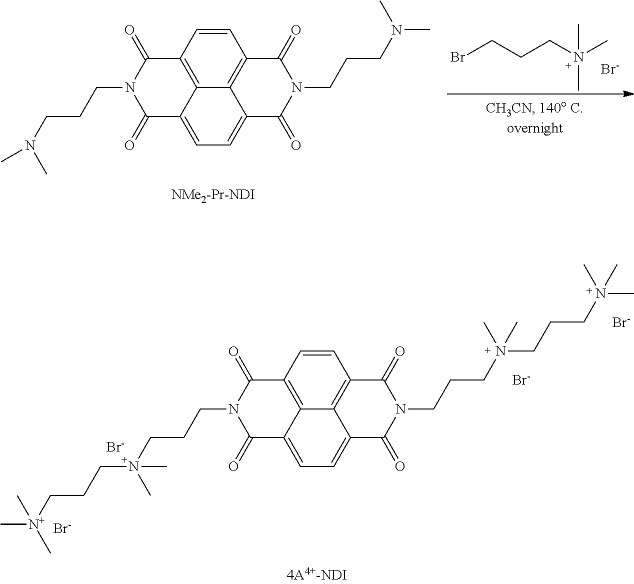

3.0 g of NMe₂-Pr-NDI prepared in Step 1 was added to 100 mL of anhydrous MeCN with stirring in a 250 mL round bottom flask in an argon atmosphere. 5 equivalents of 3-(bromopropyl) trimethylammonium bromide was added thereto with stirring. The resulting mixture was refluxed at 140° C. overnight. The resulting off-white precipitate was filtered and washed with 50 mL of acetonitrile, 50 mL of chloroform, and 30 mL of acetone. After vacuum drying, the precipitate was dissolved in a minimum amount of water and reprecipitated with acetone. While dissolution and precipitation were repeated three times, the off-white precipitate turned to light yellow to yellow. Finally, the precipitate was dried at 100° C. under vacuum to obtain 4.9 g of a final compound (yield: 69%).

¹H NMR of 4A⁴⁺-NDI: ¹H NMR (D20, 300 MHZ): δ 8.55 (s, 4H, Ar—H), δ 4.32 (t, 4H, —N_{imide}—CH₂—), δ 3.72 (t, 4H, —CH₂—N+(CH₃)₃), δ 3.59-3.52 (m, 8H, —CH₂—N+(CH₃)₂—CH₂—CH₂—CH₂—N+(CH₃)₃)), δ 3.31 (s, 18H, —N⁺(CH₃)₃), δ 3.29 (s, 12H, —N+(CH₃)₂), δ 2.47 (quint, 4H, —CH₂—CH₂—N+(CH₃)₃)), δ 2.37 (quint, 4H, —CH₂—CH₂—N+(CH₃)₂)).

ESI-MS (positive ion mode, H₂O): Observed: m/z 159.63. Calculated: m/z 159.61.

### COMPARATIVE EXAMPLE 1

### Preparation of Me-NDI

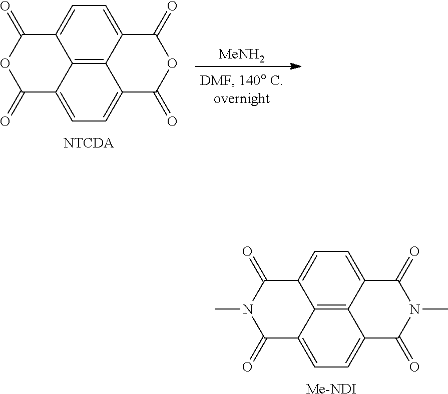

3.0 g of 1,4,5,8-naphthalenetetracarboxylic dianhydride (NTCDA) was added to 20 mL of anhydrous DMF with stirring in a 100 mL round bottom flask in an argon atmosphere. 5 equivalents of methylamine was added thereto with stirring. The resulting mixture was refluxed at 140° C. overnight. The mixture was cooled to room temperature, and Me-NDI powder was collected by filtration. The Me-NDI powder was washed with 10 mL of DMF, 50 mL of water, 50 mL of acetone, and 10 mL of diethyl ether, and the washed Me-NDI powder was dried at 100° C. under vacuum, thereby obtaining 3.13 g of a target compound (2,7-dimethylbenzo [lmn] [3,8] phenanthroline-1,3,6,8 (2H,7H) -tetraone) (Me-NDI) (yield: 95%).

¹H NMR of Me-NDI: ¹H NMR (CDCl₂, 400 MHZ): δ 8.78 (s, 4H, Ar—H), δ 3.61 (s, 6H, N_{imide}—CH₃).

### COMPARATIVE EXAMPLE 2

### Preparation of K₂BNDI

Step 1: Preparation of 2,2′-(1,3,6,8-tetraoxo-1,3,6,8-tetrahydrobenzo[lmn] [3,8]phenanthroline-2,7-diyl)diacetic Acid (H₂-BNDI)

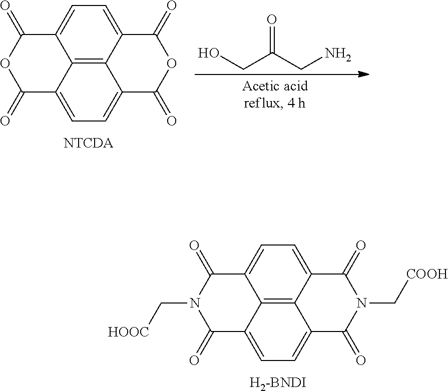

1,4,5,8-Naphthalenetetracarboxylic dianhydride (NTCDA) was added together with glycine to glacial acetic acid and the resulting mixture was refluxed for 4 hours. The mixture was cooled to room temperature, and H₂-BNDI powder was collected by filtration.

Step 2: Preparation of 2,2′-(1,3,6,8-tetraoxo-1,3,6,8-tetrahydrobenzo [lmn] [3,8]phenanthroline-2,7-diyl)diacetate Potassium (K₂-BNDI)

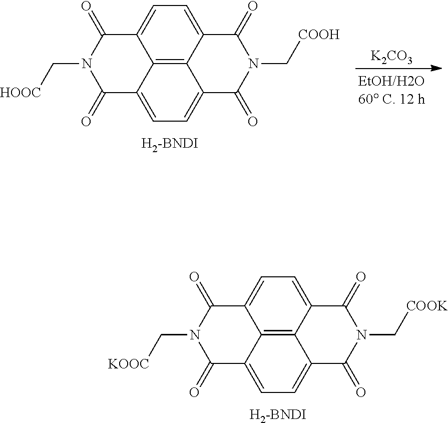

2 mM H₂-BNDI prepared in Step 1 and 2 mM potassium carbonate were added to 40 mL of water and pure ethanol at a volume ratio of 1:4 (V:V), and the mixture was stirred at 70° C. for 12 hours. The resulting precipitate was filtered and washed with ethanol. Finally, the precipitate was dried at 60° C. to obtain a target compound (K₂-BNDI) with yield of 92%.

¹H NMR of K₂-BNDI: ¹H NMR (D₂O, 400 MHZ): δ 8.54 (s, 4H, Ar—H), § 4.78 (s, 4H, N_{imide}—CH₂—)

¹³C NMR of K₂-BNDI: ¹³C NMR (D₂O, 100 MHZ): δ 174.46 (—COOK), δ 163.45 (—N—C═O), δ 131.00 (Ar—C), δ 125.77 (Ar—C), δ 125.68 (Ar—C), δ 43.77 (—CH₂—).

### COMPARATIVE EXAMPLE 3

### Preparation of 2A²⁺-NDI

Step 1: Preparation of 3,3′-(1,3,6,8-tetraoxo-1,3,6,8-tetrahydrobenzo[lmn] [3,8]phenanthroline-2,7-diyl)bis(N-ethyl-N,N-dimethylpropane-1-aminium (2A²⁺-NDI)

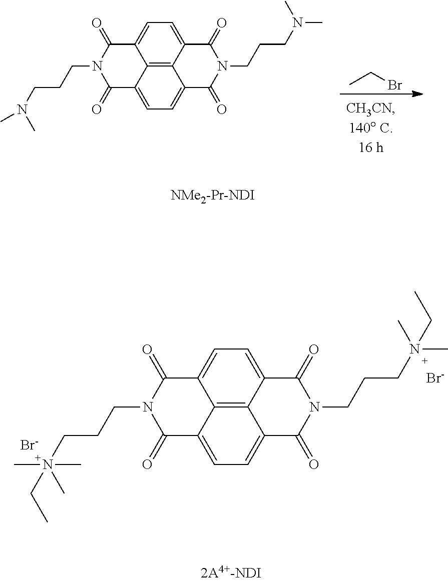

4.0 g of NMe₂-Pr-NDI prepared in Step 1 of Example 1 was added to 100 mL of anhydrous MeCN with stirring in a 250 mL round bottom flask in an argon atmosphere. 5 equivalents of 1-bromoethane was added thereto with stirring. The resulting mixture was refluxed at 140° C. for 16 hours. The resulting off-white precipitate was filtered and washed with 50 mL of acetonitrile, 50 mL of chloroform, and 30 mL of acetone. After vacuum drying, the precipitate was dissolved in a minimum amount of water and reprecipitated with acetone. While dissolution and precipitation were repeated three times, the off-white precipitate turned to light yellow to yellow. Finally, the precipitate was dried at 100° C. under vacuum to obtain 4.0 g of a target compound (2A²⁺-NDI) (yield: 85%).

¹H NMR of 2A²⁺-NDI: ¹H NMR (D20, 400 MHZ): δ 8.51 (s, 4H, Ar—H), δ 4.21 (t, 4H, —N_{imide}—CH₂—), δ 3.54-3.49 (m, 4H, —CH₂—N⁺(CH₃)₂(C₂H₅)), δ 3.43 (q, 4H, —N⁺(CH₃)₂(CH₂CH₃)), δ 3.10 (s, 12H, —N⁺(CH₃)₂(CH₂CH₃)), δ 2.32-2.21 (m, 4H, —CH₂—CH₂—CH₂), § 1.39 (t, 6H, —CH₂—CH₃)).

### Experiment Methods

### 1. Cyclic Voltammetry (CV)

Cyclic voltammetry was performed at room temperature using a three-electrode cell composed of glassy carbon (GC) having a diameter of 0.3 cm (area=0.0707 cm²) or CNT/CF as a working electrode (WE), flame-cleaned platinum wire as a counter electrode (CE), and leak-free Ag/AgCl as a reference electrode. The working electrode was polished with 50 μm alumina powder and rinsed with deionized water. All electrolyte solutions were purged with Ar gas for 10 minutes before CV measurements. An Ar atmosphere without bubbling was maintained during the CV measurements. All CV data were converted to the NHE reference using a reference of [Fe(CN)₆]³⁻/[Fe(CN)₆]⁴⁻ (E_{red}=0.358 V vs NHE).

### 2. UV-Vis-NIR Spectra

A small amount of the negative electrode electrolyte solution was taken at 0%, 10%, 25%, 50%, 75%, and 100% SOC and re-oxidized to 0% Soc, and then, the re-oxidized negative electrode electrolyte solution was transferred to a quartz cuvette and diluted with Ar-bubbled D₂O (total volume=2 mL). The cuvette was sealed and UV-Vis-NIR spectra were recorded with an air baseline in a range of 200 to 2,000 nm.

### 3. ¹H NMR Spectroscopy

0.5 mL of the negative electrode electrolyte solution was taken at 0%, 10%, 25%, 50%, 75%, and 100% SOC and re-oxidized to 0% SOC in J Young NMR tube, and sealed. Thereafter, ¹H NMR spectra were recorded.

### 4. EPR Spectroscopy

0.5 mL of the negative electrode electrolyte solution was taken at 0%, 10%, 25%, 50%, 75%, and 100% SOC and put into an EPR tube, and then, the tube was sealed with parafilm and taken out of the glove box. Then, the tube was slowly immersed in liquid N₂ and the parafilm seal was removed to evacuate trapped gas so as to prevent breakage of the tube. EPR spectra were recorded with the following experimental parameters: microwave frequency=9.4 GHz; microwave power=0.6 mW; modulation frequency=100 kHz; modulation amplitude=1 G; sweep width=200 G; time constant=0.01 ms; conversion time=15 ms; sweep time=30 s; number of scans=4; and temperature=100 K.

### EXPERIMENTAL EXAMPLE 1

### Analysis of Solubility

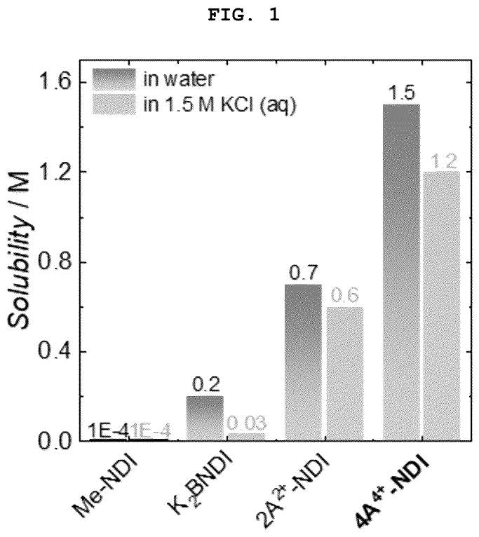

In order to analyze the solubility of the compounds of Comparative Examples and Example in water and 1.5 M KCl, the test was performed by the following method. First, the compound was dissolved in 2 mL of deionized water (DI water), and then the UV-Vis spectrum of the solution was measured. 1 mL of the solution was taken out, 1 mL of deionized water was added to reduce the concentration by half, and then the UV-Vis spectrum was measured. This process was repeated several times. A calibration curve was drawn using λ=361 nm (π-π* transition) from the UV-Vis spectrum. At this time, a UV-Vis spectrum of deionized water was used for baseline measurement. Next, the compound was separately dissolved in 1 mL of deionized water until the compound was no longer soluble at room temperature. The solution was filtered, and then the total volume of the solution was recorded. 10 μm of the filtrate was taken, and then deionized water was added to bring the volume to 2 mL. Thereafter, 1 mL of the solution was taken and fresh deionized water was added to dilute the solution to a volume of 2 mL. The UV-Vis spectrum was recorded until the absorbance at λ=361 nm was smaller than 1 a.u. The linear equation of the calibration curve was used to calculate the concentration of the solution, and the molarity equation was used to estimate the maximum solubility of the compound. The solubility in the 1.5 M KCl aqueous solution was measured in a similar manner. The results are shown in FIG. 1.

Basically, since the naphthalene core and its derivative had low solubility in water, the compound of Comparative Example 1 was substantially insoluble in water. In addition, the compound of Comparative Example 2 in which a charged functional group such as a carboxylate was added to the NDI core had a solubility of only about 25 mM in the 1 M KCl solution. The solubility 4-1 41 Comparative Example 3 to which two trimethyl ammoniumpropyl groups were attached increased to about 0.7 M in water and about 0.6 M in the KCl solution. It could be confirmed that the solubility of the compound of Example 1 to which two additional ammonium groups were added was about 1.5 M in water and 1.2 M in the KCl solution, which showed that the solubility was significantly increased compared to those of Comparative Examples.

### EXPERIMENTAL EXAMPLE 2

### Analysis of Electrochemical Properties

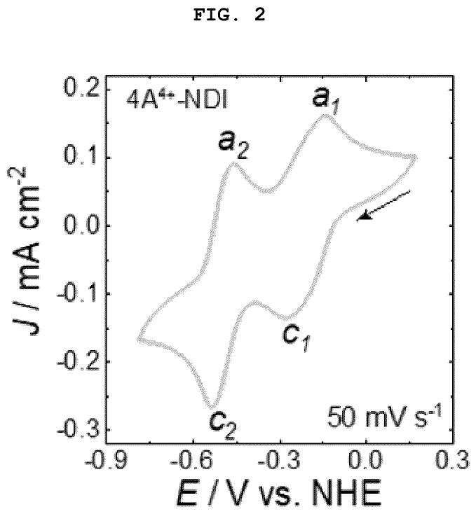

The electrochemical properties of the compound of Example were analyzed using cyclic voltammetry (CV) in a three-electrode cell composed of a KCl solution bubbled with Ar gas using glassy carbon, Pt wire, and Ag/AgCl as a working electrode, a counter electrode, and a reference electrode, respectively. The results are shown in FIG. 2. FIG. 2 shows two pairs of redox waves for a normal hydrogen electrode (NHE) at formal potentials (E⁰′) of −0.21 V and −0.50 V. As shown in the following Equations 1 and 2, the radical ion (4A⁴⁺-NDI^{•-}) was first provided and was then assigned to stepwise reduction to form the divalent anion NDI (4A⁴⁺-NDI²⁻). In FIG. 2, in Equations 1 and 2, the forwarding cathodic reactions are indicated as c₁ and c₂, and the reverse anodic reactions are indicated as a₁ and a₂, respectively.

$$4 {A}^{4 +} - NDI + {e}^{-} ⇄ 4 {A}^{4 +} - {NDI}^{* -} \qquad [ Equation 1 ]$$

$$4 {A}^{4 +} - {NDI}^{* -} + {e}^{-} ⇄ 4 {A}^{4 +} - {NDI}^{2 -} \qquad [ Equation 2 ]$$

### EXPERIMENTAL EXAMPLE 3

### Redox Flow Battery (RFB) Test-Chemical Analysis

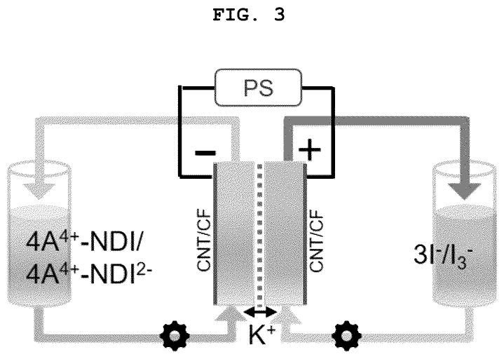

In order to perform a flow battery using the compound of Example 1, a battery was formed using a 1.5 M KCl aqueous solution as a negative electrode electrolyte solution (negolyte) and a 3I⁻/I₃⁻ redox pair as a positive electrode electrolyte solution (posolyte) (FIG. 3).

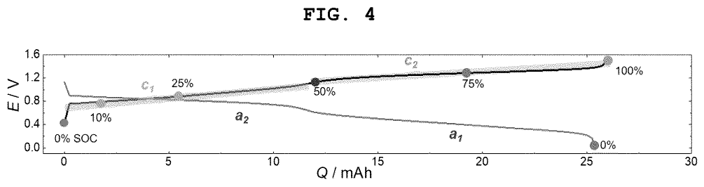

FIG. 4 shows two successive charging plateaus of galvanostatic profiles of a redox flow battery (RFB), in which c₁ and c₂ processes are the first plateau (0 to 50% stage-of-charge (SOC)) and the second plateau (50 to 100% stage-of-charge (SOC)), respectively.

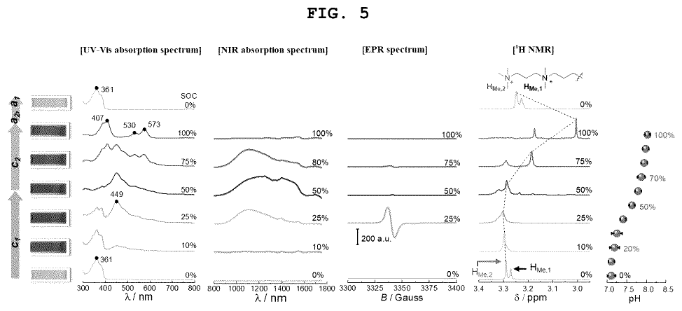

The negative electrode electrolyte solution after SOC was collected for chemical analysis of the RFB, and UV-Vis absorption spectra, near infrared radiation (NIR) absorption spectra, electron paramagnetic resonance (EPR) spectra, ¹H NMR, and solution pH were analyzed and shown in FIG. 5.

First, the results of analyzing the c₁ process of 25 to 50% SOC were as follows. As a result of analyzing the color of the electrolyte solution, the initially colorless solution exhibited yellow and reddish brown colors during the c₁ process. Through the UV-Vis spectrum, it could be confirmed that the π→π* transition band shifted from 361 nm to 449 nm at 25 to 50% SOC. A near-infrared (NIR) signal appeared at about 1,100 nm. An additional band above 1,200 nm suggests more NDI^{•-} stacking. At 25% SOC, an EPR signal appeared in a neat 50 mM solution of Example 1 without hyperfine splitting, which showed that efficient delocalization of the intermediate radical electrons. In addition, a minute EPR signal was detected at 50% SOC, indicating that the radical character was effectively nullified in the dimeric adduct.

Next, the result of analyzing the c₂ process was as follows. During the c₂ process, the electrolyte solution exhibited pink. Multiple UV-Vis absorption peaks appeared at 407, 530, and 573 nm, while the π→π* transition of the NDI^{•-} was attenuated at 100% SOC. As shown in UV-Vis and NIR spectra, the π-dimeric intermediates were still detected in the middle of the c₂ process (75 to 80% SOC). However, it could be confirmed that the EPR signal was significantly attenuated compared to that shown at 25% SOC. Through this, it could be appreciated that NDI^{•-} could be dimerized even at 75% SOC. When the charging process was completely finished, the NIR absorption and the EPR characteristics completely disappeared.

When NDI²⁻ was oxidized to become neutral during the a₂ and a₁ processes, the electrolyte solution became colorless, and the spectral signals for the two H_{Me} groups shifted back to the original positions. This demonstrates that a fully reversible redox process occurs.

In addition, it could be confirmed through pH analysis that a neutral condition of a pH of 7.0 to 8.2 was maintained throughout the entire redox process. This is in stark contrast to other redox-active organic molecules that rely on protons to reach charge neutrality during reduction, such as anthraquinone that shows a pH rise from 7 to 12.

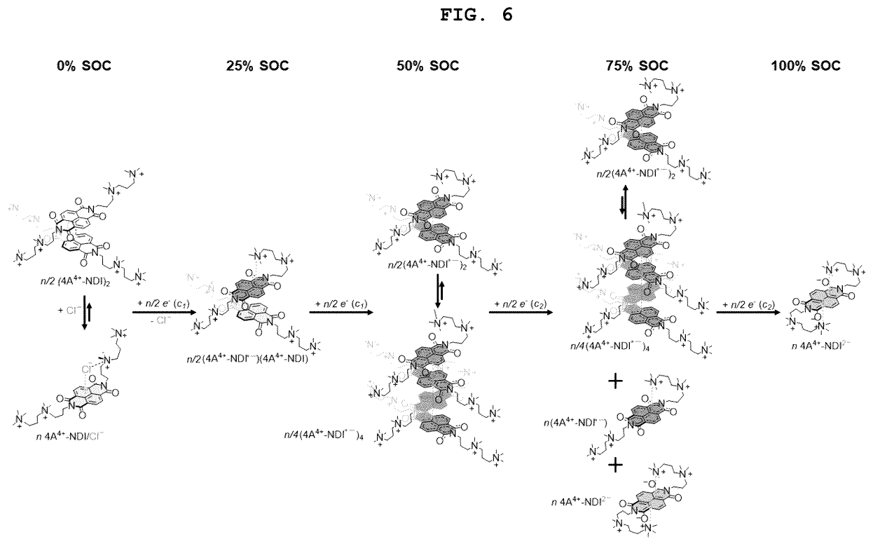

It could be confirmed through the chemical analysis that strong intramolecular interactions between the NDI and the tethered ammonium groups are critical for stabilizing the two electrons stored in the NDI core. The ¹H NMR spectra of FIG. 5 showed the intramolecular interactions and increased shielding of the ammonium methyl groups H_{Me,1} and H_{Me,2}, and in particular, the terminal ammonium group H_{Me,2} showed a notable upfield shift of Δδ=0.29 ppm at 75 to 100% SOC in contrast to the normal chemical shift of all ammonium groups at 0 to 50% SOC, which suggested that the terminal ammonium groups were in close proximity to the NDI²⁻. The interactions of the compound of Example 1 during the cathodic reaction are specifically shown in FIG. 6. The radical character in the even-numbered 4A⁴⁺-NDI clusters is effectively masked by spin-pairing across two NDI moieties. Accordingly, the compound of Example is effective in stabilizing the electrolyte because the radical character is quenched in the RFB.

### EXPERIMENTAL EXAMPLE 4

### Analysis of RFB Performance

In order to evaluate the performance of the compound of Example, a full RFB test was performed while increasing the concentration of the compound. The RFB was composed as follows.

A zero-gap flow cell was assembled with a carbon felt electrode mixed with two carbon nanotubes (CNT/CF, geometric surface area: 6 cm²) with Nafion 212 exchange membrane (thickness: 50.8 μm) sandwiched therebetween. The active area of the flow cell was 6 cm². The negative electrode electrolyte solution (negolyte) was prepared by adding the compound (×M 4A⁴⁺-NDI) of Example 1 to a 1.5 M KCl aqueous solution. The positive electrode electrolyte solution (posolyte) was prepared by adding 20 mL of 3.1×M NH₄I to a 1.5 M KCl aqueous solution. The pH values of all electrolyte solutions were 7.0 to 7.6. The reservoirs of the electrolyte solutions were purged with Ar gas for 30 minutes and sealed. As peristaltic pumps for allowing the redox-active materials to flow, model LabV1 available from Shenchen and/or model 77202-60 available from Masterflex were used. Norprene Tubing (Φ=16) of Masflex was used to flow the liquids. The solutions were circulated through the flow cell at a flow rate of 40 mL·min⁻¹. The flow cells were galvanostatically cycled at room temperature within the voltage window of 0.0 to 1.8 V at current densities ranging from 5 to 50 mA·cm⁻².

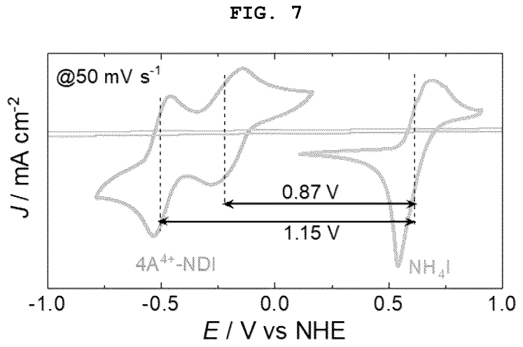

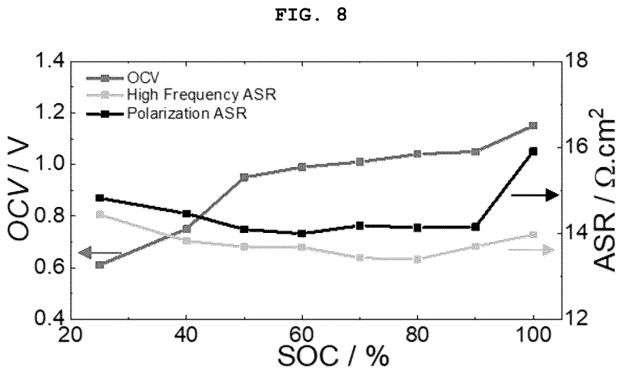

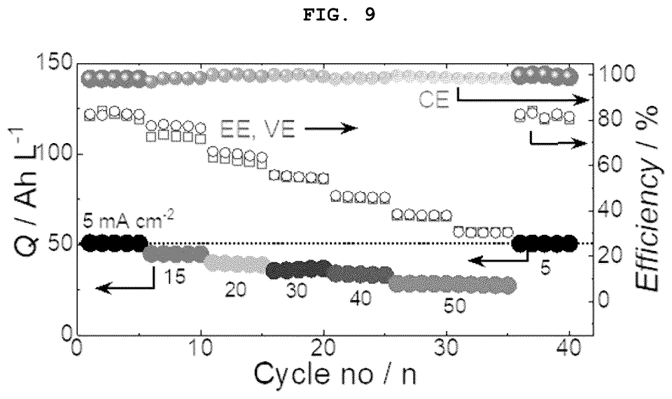

As a result of observing the c₁ and c₂ redox waves of 4A4+-NDI in CV, the two potential differences between the negative electrode electrolyte solution and the positive electrode electrolyte solution were 0.87 V and 1.15 V (FIG. 7). At 1.0 M of 4A⁴⁺-NDI corresponding to an electron concentration of 2.0 M, the RFB delivered a capacity of 52.57 Ah·L⁻¹ at a current density of 5 mA·cm⁻², indicating about 98% efficiency of the two-electron redox process (FIG. 8). Coulombic efficiency (CE) was 98.23% for the 1^{st} cycle, and both voltage efficiency (VE) and energy efficiency (EE) reached about 82% at 5 mA·cm⁻² (FIG. 9).

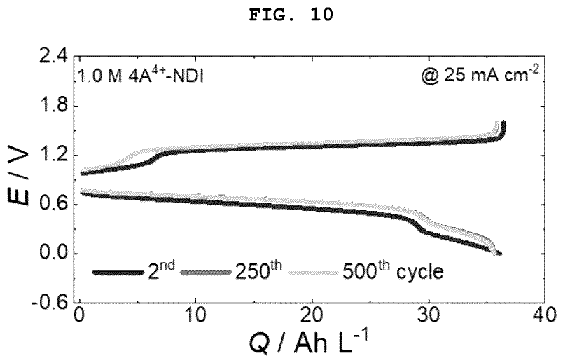

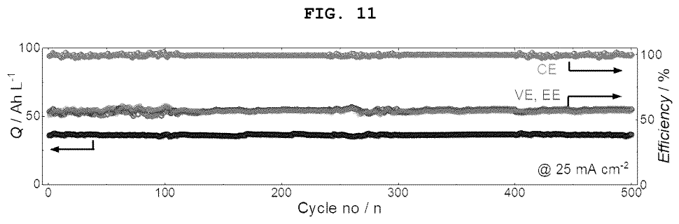

In the long-term cycling test, it was confirmed that about 98% of the capacity was maintained at 25 mA·cm⁻² for 500 cycles for 45 days, the capacity decay per cycle was 0.004%, and the capacity decay per day was 0.044% (FIGS. 10 and 11).

It could be confirmed through the above analysis that battery characteristics such as voltage efficiency and energy efficiency of the redox flow battery were excellently improved by using the compound of Example.

The present disclosure relates to a novel naphthalene diimide compound, an electrolyte solution for a redox flow battery containing the same, and a redox flow battery containing the same. The naphthalene diimide compound according to one embodiment exhibits a robust redox behavior, has high solubility in water, and has an advantage of being able to operate at neutral pH. In addition, since the naphthalene diimide compound according to one embodiment may improve the energy density by combining two electrons, it is useful as an additive for an electrolyte solution for a redox flow battery.

Hereinabove, one embodiment has been described in detail through Examples and Experimental Examples, but the scope of one embodiment is not limited to a specific embodiment, and should be interpreted according to the appended claims.

1. A naphthalene e diimide compound represented by the following Chemical Formula 1: 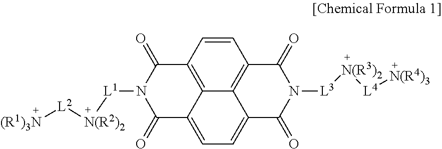
in Chemical Formula 1,
L₁, L₂, L₃, and L₄ are each independently a bond or a C₁₋₂₀ alkylene group; and
when R₁, R₂, R₃, and R₄ are each independently hydrogen, a C₁₋₂₀ alkyl group, a C₂₋₂₀ alkenyl group, a C₅₋₂₀ cycloalkyl group, or a C₆₋₂₀ aryl group, or R₁, R₂, R₃, or R₄ is an alkyl group, R₁, R₂, R₃, and R₄ may be linked together with the N atoms to which they are bonded to form a heterocycloalkyl.

2. The naphthalene diimide compound of claim 1, wherein L₁, L₂, L₃, and L₄ are each independently a bond or a C₁₋₁₀ alkylene group; and
when R₁, R₂, R₃, and R₄ are each independently hydrogen, a C₁₋₁₀ alkyl group, a C₂₋₁₀ alkenyl group, a C₅₋₁₀ cycloalkyl group, or a C₆₋₁₀ aryl group, or R₁, R₂, R₃, or R₄ is an alkyl group, R₁, R₂, R₃, and R₄ may be linked together with the N atoms to which they are bonded to form a heterocycloalkyl.

3. The naphthalene diimide compound of claim 1, wherein L₁, L₂, L₃, and L₄ are each independently a bond or a C₁₋₅ alkylene group; and
R₁, R₂, R₃, and R₄ are each independently hydrogen, a C₁₋₅ alkyl group, or a C₂₋₅ alkenyl group.

4. The naphthalene diimide compound of claim 1, wherein R₁, R₂, R₃, and R₄ are each independently a C₁₋₃ alkyl group.

5. The naphthalene diimide compound of claim 1, wherein the naphthalene diimide compound is represented by the following Chemical Formula 1-1:

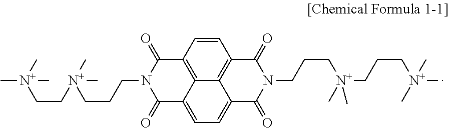

6. An electrolyte solution for a redox flow battery comprising the naphthalene diimide compound according to claim 1.

7. The electrolyte solution for a redox flow battery of claim 6, further comprising a solvent.

8. The electrolyte solution for a redox flow battery of claim 7, wherein the solvent is an aqueous solvent, a non-aqueous solvent, an ionic liquid, or a mixture of two or more thereof.

9. The electrolyte solution for a redox flow battery of claim 8, wherein the aqueous solvent is one or more selected from the group consisting of potassium chloride (KCl), sodium chloride (NaCl), potassium hydroxide (KOH), sodium hydroxide (NaOH), potassium bromide (KBr), zinc chloride (ZnCl₂), ammonium chloride (NH₄Cl), sulfuric acid (H₂SO₄), hydrochloric acid (HCl), phosphoric acid (H₃PO₄), and methanesulfonic acid (CH₃SO₄);
the non-aqueous solvent is one or more selected from the group consisting of dimethylacetamide (DMA), acetonitrile (MeCN), dimethyl carbonate, diethyl carbonate, methyl ethyl carbonate, dimethylformamide, propylene carbonate, ethylene carbonate, N-methyl-2-pyrrolidone, fluoroethylene carbonate, gamma-butyrolactone, dimethyl sulfoxide, diethylene glycol dimethyl ether, triethylene glycol dimethyl ether, tetraethylene glycol dimethyl ether, acetone, acetylacetone, 1,4-dioxane, 1,2-dimethoxyethane, dichloromethane, 1,2-dichloroethane, nitrobenzene, nitromethane, tetrahydrofuran, 2-methyltetrahydrofuran, 2,4-dimethyltetrahydrofuran, methoxybenzene, diglyme, triglyme, tetraglyme, 4-methyl-2-pentanone, propionitrile, butyronitrile, isobutyronitrile, benzonitrile, sulfolane, dimethylthioformamide, methyl acetate, ethyl acetate, ethanol, and methanol; and
the ionic liquid is an ammonium, imidazolium, morpholinium, phosphonium, piperidinium, pyridinium, pyrrolidinium, or sulfonium-based ionic liquid.

10. The electrolyte solution for a redox flow battery of claim 6, further comprising a supporting electrolyte.

11. The electrolyte solution for a redox flow battery of claim 6, further comprising an electrode active material.

12. The electrolyte solution for a redox flow battery of claim 11, wherein the electrode active material contains a 2-valent to 5-valent vanadium-based compound.

13. The electrolyte solution for a redox flow battery of claim 12, wherein the 2-valent to 5-valent vanadium-based compound comprises one or two or more selected from the group consisting of V²⁺, V³⁺, VO²⁺, and VO₂⁺.

14. A redox flow battery comprising the electrolyte solution for a redox flow battery according to claim 6.

15. The redox flow battery of claim 14, wherein the redox flow battery comprises:
a positive electrode cell comprising a positive electrode and a positive electrode electrolyte solution;
a negative electrode cell comprising a negative electrode and a negative electrode electrolyte solution; and
a separator disposed between the positive electrode cell and the negative electrode cell, and
the negative electrode electrolyte solution is the electrolyte solution for a redox flow battery.
